(19) United States
(12) Patent Application Publication
(10) Pub. No.: US 2026/0009006 A1
(43) Pub. Date: Jan. 8, 2026

(54) PRESERVATIVE COMPOSITION FOR BIOLOGICAL MARKERS AND BIOLOGICAL SAMPLES AND METHODS OF USE

(71) Applicant: SiO2 Medical Products, LLC, Auburn, AL (US)

(72) Inventors: Christopher Weikart, Auburn, AL (US); Robert S. Abrams, Auburn, AL (US); Robert Pangborn, Auburn, AL (US)

(21) Appl. No.: 19/108,660

(22) PCT Filed: Sep. 6, 2023
(86) PCT No.: PCT/US2023/032079

§ 371 Date: Mar. 4, 2025

Related U.S. Application Data
(60) Provisional application No. 63/404,174, filed on Sep. 6, 2022.

(51) Int. Cl. C12N 5/00

(52) U.S. Cl. CPC C12N 5/526

(57) ABSTRACT

This disclosure is directed to biological marker and cell preservative compositions. Methods of preserving biological markers and/or cells in a blood or other biological sample, and kits for preserving biological markers and/or cells in a blood or other biological sample are also described.

### CROSS-REFERENCE TO RELATED APPLICATION

This application claims the benefit of, priority to, and incorporates by reference in its entirety U.S. Provisional Application No. 63/404,174, filed Sep. 6, 2022.

### FIELD OF THE DISCLOSURE

The disclosure relates to compositions, methods and kits for preserving biological markers and/or cells in blood or other biological samples.

### BACKGROUND OF THE DISCLOSURE

Multi-omics, variously called integrated omics, pan-omics, and trans-omics, aims to combine two or more omics data sets to aid in data analysis, visualization and interpretation to determine the mechanism of a biological process. Multi-omics efforts have taken center stage in biomedical research leading to the development of new insights into biological events and processes. In the last decade, the application of different individual omic studies (e.g., genomics, epigenomics, transcriptomics, proteomics, metagenomics) that aimed at understanding a particular problem in human disease have been successful to a great extent (Karczewski and Snyder, 2018). These studies generate a plethora of data, which, with careful integration under a suitable statistical and mathematical framework, can help to solve broader queries pertaining to basic and applied areas of biology. Multi-omics aims to identify molecular markers associated with biological processes by revealing the regulatory units across diverse omics layers (e.g., obtained from DNA, RNA, proteins, metabolites, etc.). Multi-omics provides insights in understanding the mechanisms underlying biological processes and molecular functions, interactions and cellular fate, whether in vivo or in vitro, to reveal molecular phenotypes. Multi-omics can support discovery of predictive or prognostic biomarkers and/or potentially repurposed and novel drug targets in the era of precision medicine. Thus, the ultimate purpose of applied multi-omics is to increase the diagnostic yield for health and improve disease prognosis via robust understanding of genotype-to-phenotype relationship.

Tackling multi-omics first requires preserving cells long enough to extract and purify the biological markers without degradation so they can be used in omics studies. While multi-omics workflows are rapidly emerging as standard in biological analysis, each analyte often requires its own specialized preservative, making it difficult to obtain gene expression, epigenomic, and metabolomic information derived from both cell-free and cell type-specific sources within the same sample. It is often the case that the blood and other biological samples are collected at a different location and at a very different time than where and when they are analyzed. For this reason, after blood or other biological samples are collected, they need to be stored and transported before they can be analyzed. Due to the degradation of nucleic acids, proteins and metabolites that occurs in blood and other biological samples after they are collected, there is a need for compositions and methods that preserve these biological markers that are present in samples in order to ensure that the biological markers of the samples are of high quality at the time they are analyzed. An ideal formulation is one that keeps the cells alive unlike other preservatives that cross-link and lyse the cells leading to hemolysis and contamination. Live cells are advantageous for preserving certain biological markers at room temperature without cold storage.

Applicants demonstrate herein a proprietary formulation that preserves cells while alive as well as preserves both cellular and cell free analytes (proteins, nucleic acids, lipids, and metabolites) that is compatible with downstream workflows, enabling extraction of the maximum amount of data from a given sample and seamless integration of diverse data sets to achieve a holistic understanding of health and disease.

### SUMMARY OF THE DISCLOSURE

This disclosure is directed in various aspects to biological markers and cell preservative compositions, kits containing those compositions and methods of using the compositions and kits.

In a first aspect, the disclosure is directed to a biological marker and cell preservative composition comprising:
a. one or more enzyme inhibitors;
b. optionally one or more metabolic inhibitors;
c. optionally one or more cell surface remodeling polymers;
d. optionally one or more agents selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them; and
e. optionally polypropylene glycol (PPG);
   wherein at least one cell surface remodeling polymer (c) or agent (d) is present.

In a second aspect, the disclosure is directed to a biological marker and cell preservative composition comprising:
a. one or more enzyme inhibitors;
b. optionally one or more metabolic inhibitors;
c. one or more cell surface remodeling polymers; and
d. optionally polypropylene glycol (PPG).

In a third aspect, the disclosure is directed to a combination of a preservative composition of the disclosure and a biological sample.

In a fourth aspect, the disclosure is directed to a method for preserving biological markers and/or cells in a biological sample comprising the steps of combining a preservative composition of the disclosure and the biological sample.

In a fifth aspect, the disclosure is directed to a kit for preserving biological markers and/or cells in a biological sample comprising:
a. a preservative composition of this disclosure; and
b. optionally, instructions for use of the preservative composition.

In a sixth aspect, the disclosure is directed to a kit for preserving biological markers and/or cells in a biological sample comprising:
a. a blood or other biological sample collection tube optionally containing an anticoagulant;
b. a syringe containing a preservative composition of this disclosure; and
c. optionally, a needle attachable to said syringe.

In a seventh aspect, the disclosure is directed to a kit for preserving biological markers and/or cells in a biological sample comprising:
a. a blood or other biological sample collection tube optionally containing an anticoagulant; and
b. a sealed ampule, containing a preservative of this disclosure, wherein said ampule comprises a removable closure and wherein said ampule is configured to receive a dispensing means upon removal of the closure by a user.

In some embodiments, the biological sample is derived from a bodily fluid. In some embodiments the bodily fluid is blood. In some embodiments, the biological marker are nucleic acids.

In some embodiments, the nucleic acids are cell free (“cf”) DNA. In other embodiments of the disclosure, the nucleic acids are cellular (i.e., genomic or “g”) DNA.

In some embodiments, the nucleic acids are cell free (“cf”) RNA. In other embodiments of the disclosure, the nucleic acids are cellular (i.e., genomic or “g”) RNA.

In some embodiments, the cells are stem cells, bone cells, blood cells (e.g., red blood cells and/or white blood cells), muscle cells, fat cells, skin cells, nerve cells, endothelial cells, sex cells, pancreatic cells, cancer cells, tumor cells, or circulating tumor cells. In some embodiments, the cells are lab-derived or modified cells.

### BRIEF DESCRIPTION OF DRAWINGS

FIG. 1 depicts a workflow for blood sample processing and extraction, isolation

and analysis of circulating free DNA (cfDNA), guide DNA (gDNA) and RNA. [cfDNA=circulating free DNA; gDNA=guide DNA]

FIG. 2 shows a graph depicting hemolysis of red blood cells of blood samples stored over 30 days in a preservative composition of this disclosure (Comp 1) compared to EDTA and Alternative Composition 1 (Alt Comp 1). Blood samples stored in Comp 1 exhibited little hemolysis compared to samples stored in EDTA and Alt Comp 1.

FIGS. 3A-3C show graphs depicting the average blood volume (FIG. 3A), average plasma volume (FIG. 3B) and average percent plasma (% Plasma, FIG. 3C) of blood samples stored in a preservative composition of this disclosure (Comp 1) compared to Alternative Composition 1 (Alt Comp 1) and EDTA. Blood samples stored in Comp 1 exhibited increased plasma volume compared to samples stored in EDTA and Alt Comp 1.

FIGS. 4A-4C show graphs depicting the purity and integrity of circulating free DNA (cfDNA) isolated from biological samples stored in a preservative composition of this disclosure (Comp 1) compared to Alternative Composition 1 (Alt Comp 1). Comp 1 preserved cfDNA integrity over time compared to Alt Comp 1.

FIGS. 5A-5C show graphs depicting the purity (FIG. 5A), average yield (FIG. 5B) and DNA integrity (FIG. 5C) of guide DNA (gDNA) isolated from biological samples stored over 30 days in a preservative composition of this disclosure (Comp 1) compared to EDTA. Biological samples stored in Comp 1 exhibited a similar performance to EDTA.

FIGS. 6A-6D show graphs depicting the average yield (FIG. 6A), purity (FIG. 6B) and integrity (FIG. 6C) of RNA isolated from biological samples stored over 10 days in a preservative composition of this disclosure (Comp 1) compared to Alternative Composition 2 (Alt Comp 2). Comp 1 preserved RNA integrity over time compared to Alt Comp 2 (n=3). FIG. 6D depicts a BioAnalyzer RNA trace representation of a single donor.

FIG. 7 describes the temperature stability of the preservative composition of this disclosure (Comp 1).

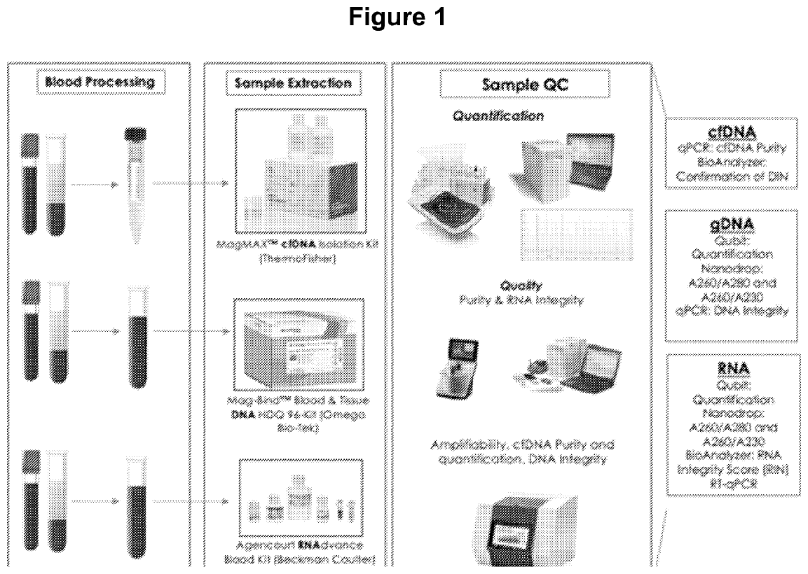

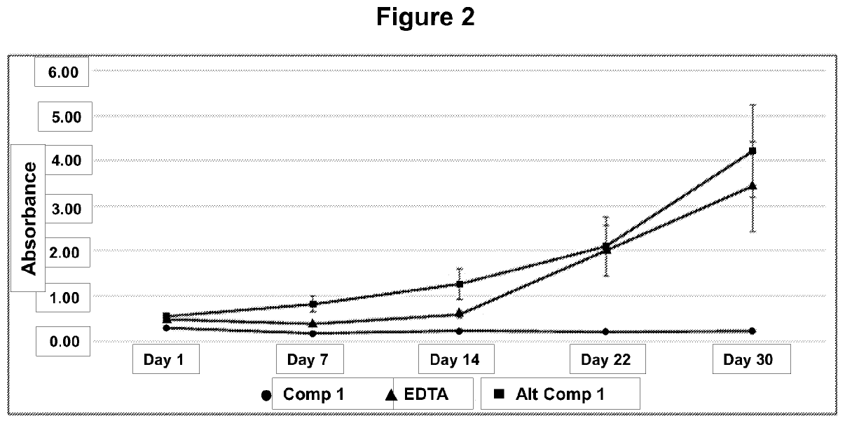

### EXEMPLARY EMBODIMENTS

Exemplary embodiments of the disclosure include:
1. A preservative composition comprising:
   a. one or more enzyme inhibitors;
   b. optionally one or more metabolic inhibitors;
   c. optionally one or more cell surface remodeling polymers;
   d. optionally one or more agents selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them; and
   e. optionally polypropylene glycol (PPG);
      wherein at least one cell surface remodeling polymer (c) or agent (d) is present.
2. The preservative composition according to embodiment 1, wherein no osmotic agent is present.
3. The preservative composition according to any one of embodiments 1 or 2, wherein no plasma expander is present.
4. The preservative composition according to any one of embodiments 1-3, wherein the one or more optional cell surface remodeling polymer(s) is present.
5. The preservative composition according to any one of embodiments 1-3, wherein the one or more optional agent(s) is not present.
6. The preservative composition according to any one of embodiments 1-5, wherein the one or more enzyme inhibitor(s) is present in the preservative compositions in an amount of about 0.5% to about 30%, about 1% to about 10%, about 1.5% to about 5%, or about 1.5% to about 2.5% by weight of the composition.
7. The preservative composition according to embodiment 6, wherein the one or more enzyme inhibitor(s) is selected form the group consisting of ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, and glucaric acid, or salts of any of them.
8. The preservative composition according to embodiment 7, wherein the enzyme inhibitor is ethylenediaminetetraacetic acid (EDTA) or salts thereof.
9. The preservative composition according to embodiment 7, wherein the EDTA or salts thereof is present in an amount of about 1.5% to about 2.5% by weight of the composition.
10. The preservative composition according to any one of embodiments 1-9, wherein the one or more optional metabolic inhibitor(s) is sodium azide, thimerosal, proclin or chlorohexidine.
11. The preservative composition according to embodiment 1-4 and 6-10, wherein the one or more optional agent(s) is a Ficoll.
12. The preservative composition according to embodiment 11, wherein the Ficoll is Ficoll 400.
13. The preservative composition according to any one of embodiments 1-12, wherein the optional cell surface remodeling polymer is a surfactant.
14. The preservative composition according to embodiment 13, wherein the surfactant is a poloxamer.
15. The preservative composition according to embodiment 14, wherein the poloxamer is selected from one or both of poloxamer p188 and poloxamer p407.
16. The preservative composition according to embodiment 15, wherein the poloxamer is poloxamer p188.
17. The preservative composition according to embodiment 15, wherein the poloxamer is poloxamer p407.
18. The preservative composition according to embodiment 15, wherein the poloxamer is a combination of poloxamer p188 and poloxamer p407.
19. The preservative composition according to any one of embodiments 14-18, wherein the poloxamer(s) is present in an amount from about 10% to about 40%, about 10% to about 35%, about 10% to about 25%, about 10% to about 20%, about 15% to about 20%, or about 15% by weight or 30% by weight of the composition.
20. The preservative composition according to embodiment 19, wherein each of the poloxamer p188 and poloxamer p407 are present in an amount of about 15% by weight of the composition.
21. The preservative composition according to any one of embodiments 1-20, wherein the optional polypropylene glycol (PPG) is present in an amount of about 0.1% to about 10% by weight of the composition.
22. A preservative composition comprising:
   a. one or more enzyme inhibitors;
   b. optionally one or more metabolic inhibitors;
   c. one or more cell surface remodeling polymers; and
   d. optionally polypropylene glycol (PPG).
23. The preservative composition according to embodiment 22, wherein no osmotic agent is present.
24. The preservative composition according to any one of embodiments 22 or 23, wherein no plasma expander is present.
25. The preservative composition according to any one of embodiments 22-24 where no agent is selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them is present.
26. The preservative composition according to any one of embodiments 22-25, wherein the one or more enzyme inhibitor(s) is present in the preservative compositions in an amount of about 0.5% to about 30%, about 1% to about 10%, about 1.5% to about 5%, or about 1.5% to about 2.5% by weight of the composition.
27. The preservative composition according to embodiment 26, wherein the one or more enzyme inhibitor(s) is selected form the group consisting of ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, and glucaric acid, or salts of any of them.
28. The preservative composition according to embodiment 27, wherein enzyme inhibitor is ethylenediaminetetraacetic acid (EDTA) or salts thereof.
29. The preservative composition according to embodiment 28, wherein the EDTA or salts thereof is present in an amount of about 1.5% to about 2.5% by weight of the composition.
30. The preservative composition according to embodiment 22-29, wherein the one or more optional metabolic inhibitor(s) is sodium azide, thimerosal, proclin or chlorohexidine.
31. The preservative composition according to embodiment 22-30, wherein the cell surface remodeling polymer is a surfactant.
32. The preservative composition according to embodiment 31, wherein the surfactant is a poloxamer.
33. The preservative composition according to embodiment 32, wherein the poloxamer is selected from one or both of poloxamer p188 and poloxamer p407.
34. The preservative composition according to embodiment 33, wherein the poloxamer is poloxamer p188.
35. The preservative composition according to embodiment 33, wherein the poloxamer is poloxamer p407.
36. The preservative composition according to embodiment 33, wherein the poloxamer is a combination of poloxamer p188 and poloxamer p407.
37. The preservative composition according to embodiment 33-36, wherein the poloxamer(s) is present in an amount from about 10% to about 40%, about 10% to about 35%, about 10% to about 25%, about 10% to about 20%, about 15% to about 20%, or about 15% by weight or 30% by weight of the composition.
38. The preservative composition according to embodiment 37, wherein each of the poloxamer p188 and poloxamer p407 are present in an amount of about 15% by weight in the composition.
39. The preservative composition according to any one of embodiments 22-38, wherein the optional polypropylene glycol (PPG) is present in an amount of about 0.1% to about 10% by weight of the composition.
40. A preservative composition comprising:
   a. 1.57 wt % EDTA or salts thereof;
   b. 15.00 wt % poloxamer p188; and
c. 15.00 wt % poloxamer p407.
41. A preservative composition comprising:
   a. 2.5 wt % EDTA or salts thereof;
   b. 15.00 wt % poloxamer p188; and
   c. 15.00 wt % poloxamer p407.
42. The preservative composition according to any one of embodiments 40 or 41, wherein no osmotic agent, plasma expander or an agent selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them is present.
43. The preservative composition according to any one of embodiments 1-42, that is in the form of a lyophilized dry powder.
44. The preservative composition according to any one of embodiments 1-42, that is in the form of an aqueous solution.
45. A combination of a preservative composition according to any one of embodiments 1-44 and a biological sample.
46. The combination according to embodiment 45, wherein the biological sample is a cell or tissue sample.
47. The combination according to embodiment 45, wherein the biological sample is derived from bodily fluids.
48. The combination according to embodiment 47, wherein the bodily fluid is whole blood or one of more fractions thereof.
49. The combination according to embodiment 45, wherein the biological sample comprises stem cells, bone cells, blood cells, muscle cells, fat cells, skin cells, nerve cells, endothelial cells, sex cells, pancreatic cells, cancer cells, tumor cells, or circulating tumor cells.
50. The combination according to embodiment 45, wherein the biological sample comprises a biological marker selected from a nucleic acid, a protein, a peptide, a metabolite, or a combination thereof.
51. The combination according to embodiment 50, wherein the biological sample comprises a nucleic acid selected from RNA, DNA, or a combination thereof.
52. The combination according to embodiment 51, wherein the nucleic acid is cell-free RNA, cell-free DNA, or a combination thereof.
53. The combination according to embodiment 51, wherein the nucleic acid is cellular RNA, cellular DNA, or a combination thereof.
54. The combination according to any one of embodiments 45-53, wherein the ratio of the preservative composition to the biological sample is from about 1:10 to about 1:1 v/v.
55. The combination according to embodiment 54, wherein the ratio of the preservative composition to the biological sample is from about 1:5 to about 1:4 v/v.
56. The combination according to any one of embodiments 49-55, wherein the composition is capable of preserving the nucleic acids and/or cells in a biological sample for at least 2 weeks at ambient temperature.
57. A method for preserving one or both of biological markers and cells in a biological sample comprising the steps of combining a preservative composition according to any one of embodiments 1-44 and the biological sample.
58. The method according to embodiment 57, wherein the biological sample is a cell or tissue sample.
59. The method according to embodiment 57, wherein the biological sample is derived from bodily fluids.
60. The method according to embodiment 59, wherein the bodily fluid is whole blood or one or more fractions thereof.
61. The method according to embodiment 57, wherein the biological sample comprises stem cells, bone cells, blood cells, muscle cells, fat cells, skin cells, nerve cells, endothelial cells, sex cells, pancreatic cells, cancer cells, tumor cells, or circulating tumor cells.

62. The method according to embodiment 57, wherein the biological sample comprises a biological marker selected from a nucleic acid, a protein, a peptide, a metabolite, or a combination thereof.
63. The method according to embodiment 62, wherein the biological sample comprises a nucleic acid selected from RNA, DNA, or a combination thereof.
64. The method according to embodiment 63, wherein the nucleic acid is cell-free RNA, cell-free DNA, or a combination thereof.
65. The method according to embodiment 63, wherein the nucleic acid is cellular RNA, cellular DNA, or a combination thereof.
66. The method according to any one of embodiments 57-65, wherein the ratio of the preservative composition to the biological sample is from about 1:10 to about 1:1 v/v.
67. The method according to embodiment 66, wherein the ratio of the preservative composition to the biological sample is from about 1:5 to about 1:4 v/v.
68. A kit for preserving one or both of biological markers and cells in a biological sample comprising:
   a. a preservative composition according to any one of embodiments 1-44; and
   b. optionally, instructions for use of said preservative composition.
69. The kit according to embodiment 68, wherein the biological sample is a cell or tissue sample.
70. The kit according to embodiment 68, wherein the biological sample is derived from a bodily fluid.
71. The kit according to embodiment 70, wherein the bodily fluid is whole blood or one or more fractions thereof.
72. The kit according to embodiment 68, wherein the biological sample comprises stem cells, bone cells, blood cells, muscle cells, fat cells, skin cells, nerve cells, endothelial cells, sex cells, pancreatic cells, cancer cells, tumor cells, circulating tumor cells, or a combination thereof.
73. The kit according to embodiment 68, wherein the biological sample comprises a biological marker selected from a nucleic acid, a protein, a peptide, a metabolite, or a combination thereof.
74. The kit according to embodiment 73, wherein the biological sample comprises a nucleic acid selected from RNA, DNA, or a combination thereof.
75. The kit according to embodiment 74, wherein the nucleic acid is cell-free RNA, cell-free DNA, or a combination thereof.
76. The kit according to embodiment 74, wherein the nucleic acid is cellular RNA, cellular DNA, or a combination thereof.
77. A kit for preserving one or both of biological markers and cells in a biological sample comprising:
   a. a blood or other biological sample collection tube optionally containing a predetermined amount of an optional anticoagulant;
   b. a syringe containing a predetermined amount of a preservative composition according to any one of embodiments 1-44; and
   c. optionally, a needle attachable to said syringe.
78. The kit according to embodiment 77, wherein the biological sample is a cell or tissue sample.
79. The kit according to embodiment 77, wherein the biological sample is derived from a bodily fluid.
80. The kit according to embodiment 79, wherein the bodily fluid is whole blood or one or more fractions thereof.
81. The kit according to embodiment 77, wherein the biological sample comprises stem cells, bone cells, blood cells, muscle cells, fat cells, skin cells, nerve cells, endothelial cells, sex cells, pancreatic cells, cancer cells, tumor cells, circulating tumor cells, or a combination thereof.
82. The kit according to embodiment 77, wherein the biological sample comprises a biological marker selected from a nucleic acid, a protein, a peptide, a metabolite, or a combination thereof.
83. The kit according to embodiment 82, wherein the biological sample comprises a nucleic acid selected from RNA, DNA, or a combination thereof.
84. The kit according to embodiment 83, wherein the nucleic acid is cell-free RNA, cell-free DNA, or a combination thereof.
85. The kit according to embodiment 83, wherein the nucleic acid is cellular RNA, cellular DNA, or a combination thereof.
86. A kit for preserving one of both of biological markers and cells in a biological sample comprising:
   a. a blood or other biological sample collection tube optionally containing a predetermined amount of an anticoagulant; and
   b. a sealed ampule, containing a predetermined amount of a preservative composition according to any one of embodiments 1-44, wherein said ampule comprises a removable closure and wherein said ampule is configured to receive a dispensing means upon removal of the closure by a user.
87. The kit according to embodiment 86, wherein the biological sample is a cell or tissue sample.
88. The kit according to embodiment 86, wherein the biological sample is derived from a bodily fluid.
89. The kit according to embodiment 88, wherein the bodily fluid is whole blood or one or more fractions thereof.
90. The kit according to embodiment 86, wherein the biological sample comprises stem cells, bone cells, blood cells, muscle cells, fat cells, skin cells, nerve cells, endothelial cells, sex cells, pancreatic cells, cancer cells, tumor cells, circulating tumor cells, or a combination thereof.
91. The kit according to embodiment 86, wherein the biological sample comprises a biological marker selected from a nucleic acid, a protein, a peptide, a metabolite, or a combination thereof.
92. The kit according to embodiment 91, wherein the biological sample comprises a nucleic acid selected from RNA, DNA, or a combination thereof.
93. The kit according to embodiment 92, wherein the nucleic acid is cell-free RNA, cell-free DNA, or a combination thereof.
94. The kit according to embodiment 92, wherein the nucleic acid is cellular RNA, cellular DNA, or a combination thereof.
95. The preservative composition of any one of embodiments 1-44, the combination of any one of embodiments 45-56, the method of 57-67, or the kit of any one of embodiments 68-94, wherein the amount of the biological markers is maximized.
96. The preservative composition of any one of embodiments 1-44, the combination of any one of embodiments 45-56, the method of 57-67, or the kit of any one of embodiments 68-94, wherein the preservative composition is contained within a blood collection tube.
97. The preservative composition of any one of embodiments 1-44, the combination of any one of embodiments 45-56, the method of 57-67, or the kit of any one of embodiments 68-94, wherein the blood collection tube is placed into a pouch after collection and the pouch is placed into a shipping case.

### DETAILED DESCRIPTION OF THE DISCLOSURE

### Definitions

Unless otherwise defined herein, scientific and technical terms used in this application shall have the meanings that are commonly understood by those of ordinary skill in the art. In case of conflict, the present specification, including definitions, will control.

Throughout this application and its various embodiments and aspects, the word “comprise,” or variations such as “comprises” or “comprising,” will be understood to allow the inclusion of a stated integer or group of integers but not the exclusion of any other integer or group of integers.

The term “including” or “includes” is used to mean “including but not limited to.” “Including” and “including but not limited to” are used interchangeably.

Any example(s) following the term “e.g.” or “for example” is not meant to be exhaustive or limiting to this disclosure.

Unless otherwise required by context, singular terms shall include pluralities and plural terms shall include the singular.

The articles “a”, “an” and “the” are used herein to refer to one or to more than one (i.e., to at least one) of the grammatical object of the article.

All ranges disclosed herein are to be understood to encompass any and all subranges subsumed therein. For example, a stated range of “1 to 10” should be considered to include any and all subranges between (and inclusive of) the minimum value of 1 and the maximum value of 10; that is, all subranges beginning with a minimum value of 1 or more, e.g., 1 to 6.1, and ending with a maximum value of 10 or less, e.g., 5.5 to 10. The term “about” when used in the context of the weight percentage of a component or v/v of a mixture means+/−10% of the recited number.

Each embodiment of this disclosure may be taken alone or in combination with one or more other embodiments of this disclosure.

Exemplary methods and materials are described herein. It should be understood that methods and materials similar or equivalent to those described herein can also be used in the practice or testing of the various aspects and embodiments of this disclosure. The materials, methods, and examples are illustrative only and not intended to be limiting.

In order for the disclosure to be more readily understood, certain terms are first defined. These definitions should be read in light of the remainder of the disclosure as understood by a person of ordinary skill in the art. Unless defined otherwise, all technical and scientific terms used herein have the same meaning as commonly understood by a person of ordinary skill in the art. Additional definitions are set forth throughout the detailed description.

As used herein, the term “osmotic agent” refers to an agent that produces a hypertonic or isotonic solution. Examples of osmotic agents include, but are not limited to, for example, sodium, potassium, magnesium and calcium salts, Ringer's lactate, Ringer's acetate, an amino acid, sorbitol, glycerol, mannitol, sugars such as sucrose or glucose, tartaric acid, and glucaric acid, or salts of any of them. Without wishing to be bound by theory, osmotic agents serve to alter osmotic pressure in the blood or other biological sample, leading, for example, to the release of water from the cells present in the blood or other biological sample to counteract the imbalance. This can cause, for example, the cells to shrink, thereby, making them more resistant to cell lysis which would otherwise cause the cell-free biological markers of the biological sample to be contaminated with cellular biological markers, or the cells to be less amenable to assay and analysis. Additionally, it is believed that plasma expander will enhance this effect.

As used herein, the term “hypertonic solution” refers to a solution with a solute concentration that is higher than physiologic. Examples of hypertonic solutions include, but are not limited to an about 2%, 3%, 4%, 5%, 6%, 7%, 8%, 9%, 10%, 12%, 14%, 15%, 16%, 17%, 18%, 19%, 20%, 21%, 22%, 23%, 24% and 25% (by weight) NaCl solution.

As used herein, the term “isotonic solution” refers to a solution with a solute concentration that is approximately equal to physiologic. Examples of isotonic solutions include, but are not limited to an about 0.5%, 0.7%, and 1% (by weight) NaCl solution.

As used herein, the term “enzyme inhibitor” refers to an agent that, alone or in a preservative composition of this disclosure, generates complexes with metal ions, such as calcium, magnesium, manganese or zinc, which complexes are believed to reduce blood coagulation, inhibit nucleases and/or reduce enzymatic cell lysis. Examples of enzyme inhibitors of this disclosure include but are not limited to ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, glucaric acid, or salts of any of them, including but not limited to sodium and potassium salts. Without wishing to be bound by theory, the inhibition of nucleases will prevent or reduce the degradation of cell-free nucleic acids within the biological sample. Examples of enzymes that the enzyme inhibitor of this disclosure inhibit, include, but are not limited to lysostaphin, zymolase, protease, glycanase, or other enzymes that are known to induce cell lysis, thereby acting to preserve the cells of blood or other biological samples.

As used herein, the term “metabolic inhibitor” refers to an agent that, alone or in a preservative composition of this disclosure, inhibits cellular processes, such as cellular respiration, cellular metabolism and metabolic function, which inhibition is believed to reduce the degradation of biological markers. Without wishing to be bound by theory, the metabolic inhibitors of this disclosure are believed to slow the growth of cells by inhibiting cell metabolic functions and suppressing bacterial growth, thereby reducing degradation of biological markers. Examples of metabolic inhibitors of this disclosure include, but are not limited to, sodium azide, thimerosal, proclin, or chlorohexidine.

As used herein, the term “plasma expander” refers to an agent that produces a hyperoncotic or hypertonic solution. Examples of plasma expanders include, but are not limited to glycerol, starch, protein colloids (e.g., albumin, ovalbumin, and gelatins) and non-protein colloids (e.g., hydroxyethyl starch). Without wishing to be bound by theory, plasma expanders also serve to increase osmotic pressure in the blood plasma or other biological sample, leading to the release of water from the cells to counteract the imbalance. This causes the cells to shrink, thereby, making them more resistant to cell lysis which would otherwise cause the cell-free biological markers of the biological sample to be contaminated with cellular biological markers, or the cells to be less amenable to assay and analysis.

As used herein, the term “cell surface remodeling polymer” refers to a polymer that interacts with a cell surface (e.g., by binding to a cell surface receptor, or by reacting with specific functional groups on the cell surface) in a blood or other biological sample through covalent interactions, hydrophobic interactions or electrostatic interactions. Such interactions are believed in some cases to cause the cells in the blood or other biological sample to sediment. Without wishing to be bound by theory, it is believed that the sedimentation of the cells in the biological sample and/or the interactions of the cell surface and the polymer prevents or reduces cell lysis and the subsequent release of cellular biological markers into the sample that may otherwise contaminate, for example, the cell-free biological markers or intact cells within the sample. The biological markers and/or cells can subsequently be isolated and analyzed via conventional methods known in the art. In some embodiments of this disclosure the “cell surface remodeling polymer’ is a surfactant. Examples of cell surface remodeling polymers include, but are not limited to, a copolymer of N-vinylpyrollidone (NVP) and a boronic acid, an arginylglyclaspartic acid (RGD) tripeptide polymer derivative, mung bean phytohaemagglutinin, a poloxamer, and a synthetic glycopeptide that is characterized by one or more ligands for the mannose 6 phosphate receptor (e.g., glycopepties bearing multiple serine-O-mannose-6-phosphonate (M6Pn) residues). For examples of glycopeptides bearing repeated ligands for the mannose 6 phosphate receptor, see Banik, Steven; Pedram, Kayvon; Wisnovsky, Simon; Riley, Nicholas; Bertozzi, Carolyn (2019): Lysosome Targeting Chimeras (LYTACs) for the Degradation of Secreted and Membrane Proteins. ChemRxiv. Preprint. https://doi.org/10.26434/chemrxiv.7927061.v2.

As used herein, a “Ficoll” refers to a water-soluble high molecular weight sucrose polymer that is formed from the polymerization of sucrose with epichlorohydrin. For example, Ficoll 400 and Ficoll 70.

As used herein, a “poloxamer” refers to a water-soluble triblock copolymer having a central hydrophobic chain of polyoxypropylene flanked by two hydrophilic chains of polyoxyethylene. Examples of poloxamers include, but are not limited to, poloxamer p188 and poloxamer p407.

As used herein, a “protein colloid” refers to a mixture in which one or more proteins is dispersed in solution. Examples of protein colloids include, but are not limited to albumin, ovalbumin, or gelatins. The albumin may be provided as, for example, a human serum albumin (HSA), a bovine serum albumin (BSA) or an ovalbumin. Examples of gelatins include, but are not limited, to urea-linked gelatins (e.g., Haemaccel®), succinylated gelatins (e.g., Gelofusine®), and oxypolygelatins.

As used herein, the term “a non-protein colloid” refers to a mixture in which one or more large molecules or ultramicroscopic particles are dispersed in solution. Examples of non-protein colloids include, but are not limited to, branched natural polymers of amylopectin, such as hydroxyethylated starches (HES), and polysaccharides, such as dextrans, for example, Dextran 40 and/or Dextran 70.

As used herein, a “water-soluble polymer” refers to a polymer that is soluble in aqueous solution. Examples of water-soluble polymers includes, but are not limited to a polyacrylamide, a polyacrylate, a polydextrose, a polyglycine, a polyethyleneimine, a polylysine, a polyethylene glycol, a polyvinyl pyrrolidone, a polyvinyl alcohol, a polyacrylic acid, a polymer of N-(2-hydroxypropyl) methacrylamide, a polymer of divinyl ether-maleic anhydride, a polyoxazoline, a polyphosphate, a polyphosphazene, a xanthan gum, a pectin, a chitosan derivative, a dextran, a carrageenan, a guar gum, a cellulose ether, a sodium carboxymethyl cellulose, a hydroxypropyl cellulose, a hypromellose, a hyaluronic acid, an albumin, a starch, or a starch based derivative. For further non-limiting examples of water-soluble polymers of the disclosure, see Betageri, G. V., Kadajji, V. G., Polymers, 2011, 3, pp. 1972-2009.

As used herein, the term “nucleic acid” includes both ribonucleic acid (RNA) and deoxyribonucleic acid (DNA). The RNA and/or DNA may be linear or branched, single or double stranded, or fragmented. The RNA and DNA may be cellular RNA (i.e., genomic RNA), cellular DNA (i.e., genomic DNA), cell-free RNA, cell-free DNA or combinations thereof. Nucleic acids are found in biological samples, and in particular, blood samples.

As used herein, the term “biological sample” refers to a sample obtained from a biological source, including lab-derived or lab-modified cells, that comprises biological markers and/or cells. Biological samples may be cell, culture or tissue samples. Additionally, biological samples may be derived from bodily fluids, such as, for example, blood, plasma, serum, urine, saliva, stool, breast milk, tears, sweat, cerebral spinal fluid, synovial fluid, semen, vaginal fluid, ascitic fluid, amniotic fluid, or cell culture media.

As used herein, the term “preservative” refers to a composition that is added to a biological sample that inhibits, prevents, or slows the degradation of the biological markers and/or cell lysis in that sample.

As used herein, the term “treated biological sample” refers to a biological sample that has been combined with a preservative composition of this disclosure.

As used herein, the term “cells” refers to any cell that may be found in blood or other biological samples. Types of cells include, but are not limited to stem cells, bone cells, blood cells (e.g., red blood cells or white blood cells), muscle cells, fat cells, skin cells, nerve cells, endothelial cells, sex cells, pancreatic cells, cancer cells, tumor cells, circulating tumor cells (CTCs) and lab derived and/or modified cells.

### Preservative Compositions of the Disclosure

The compositions of this disclosure are useful in the preservation and stabilization of biological markers and/or cells in biological samples. When the preservative compositions of the disclosure are added to a biological sample containing biological markers and/or cells, the degradation of the biological markers and/or cell lysis in that sample is reduced, slowed or prevented, as compared to untreated biological samples, allowing for the subsequent isolation and more accurate analysis of the biological markers and/or the cells in the sample via conventional techniques known in the art, particularly high throughput techniques. Additionally, the preservative compositions of the disclosure inhibit, slow, or reduce cell lysis, allowing the cell free biological markers in the sample to remain more consistent in amount and character over prolonged periods of time. The reduction of cell lysis in treated biological samples according to this disclosure also reduces the release of nucleases, thereby further preventing or reducing degradation of nucleic acids and/or cells within the sample. The biological markers that can be preserved by the compositions of the disclosure include nucleic acids, proteins, peptides and metabolites. The nucleic acids that can be preserved by the compositions of the disclosure include RNA, DNA or combinations thereof. The RNA and DNA can be cellular or cell-free or combinations thereof, i.e., cellular RNA, cellular DNA, cell-free RNA, cell-free DNA, or combinations thereof. Preferably, the DNA and/or RNA is cell-free DNA and/or RNA. The proteins, peptides and metabolites can be cellular or cell-free or combinations thereof. The cells, whose lysis is reduced using the compositions and methods of this disclosure, can be, without limitation, stem cells, bone cells, blood cells, muscle cells, fat cells, skin cells, nerve cells, endothelial cells, sex cells, pancreatic cells, cancer cells, tumor cells, circulating tumor cells and lab-derived or modified cells.

In a first aspect, the disclosure is directed to a biological marker and cell preservative composition comprising:
a. one or more enzyme inhibitors;
b. optionally one or more metabolic inhibitors;
c. optionally one or more cell surface remodeling polymers;
d. optionally one or more agents selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them; and;
e. optionally polypropylene glycol (PPG);
   wherein at least one cell surface remodeling polymer (c) or agent (d) is present.

In some embodiments of the first and the other aspects of the disclosure (without limitation the second through seventh aspects referred to herein), no osmotic agent is present.

In some embodiments of the first and the other aspects of the disclosure, no plasma expander is present.

In some embodiments of the first and the other aspects of the disclosure, the one or more enzyme inhibitor(s) is present in the preservative compositions of the disclosure in an amount of about 0.5% to about 30% by weight, in some aspects in an amount of about 0.5% to about 5% by weight and in other aspects from about 1% to about 30% by weight, of the composition. In some embodiments, the enzyme inhibitor(s) is present in an amount of about 1% to about 20% by weight of the composition. In other embodiments, the enzyme inhibitor is present in an amount of about 1% to about 10% by weight of the composition.

In some embodiments of the first and the other aspects of the disclosure, the one or more enzyme inhibitor(s) is ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, glucaric acid, or salts of any of them. The salts include, but are not limited to, sodium and potassium salts or mixtures thereof.

In some embodiments of the first and the other aspects of the disclosure, the one or more optional metabolic inhibitor(s) is present in the preservative compositions of the disclosure in an amount of about 0.01% to about 10% by weight of the composition. In some embodiments, the optional metabolic inhibitor is present in an amount of about 0.01% to about 5% by weight of the composition. In some embodiments, the optional metabolic inhibitor is present in an amount of about 0.01% to about 2% by weight of the composition.

In some embodiments of the first and the other aspects of the disclosure, the one or more optional metabolic inhibitor(s) is sodium azide, thimerosal, proclin or chlorohexidine.

In some embodiments of the first and the other aspects of the disclosure, the one or more optional agent(s) is a Ficoll. In some embodiments, the Ficoll is a Ficoll-400.

Without wishing to be bound by theory, a Ficoll serves as a crowding agent, forcing cells out of solution thereby preventing or reducing cell lysis and the subsequent degradation of the cells or release of cellular biological markers into the sample that may otherwise contaminate, for example, the cell-free biological markers within the sample. The biological markers and/or cells can then subsequently be isolated and more accurately analyzed via conventional methods known in the art.

In some embodiments of the first and the other aspects of the disclosure, the Ficoll is present in an amount of about 10% to about 50% by weight of the composition. In some embodiments, the one or more agents are present in an amount of about 10% to about 40% by weight, or from about 15% to about 35% by weight, or from about 20% to about 30% by weight of the composition.

In some embodiments of the first and the other aspects of the disclosure, the one or more cell surface remodeling polymer(s) is selected from the group consisting of copolymer of N-vinylpyrollidone (NVP) and a boronic acid, an arginylglyclaspartic acid (RGD) tripeptide polymer derivative, mung bean phytohaemagglutinin, and a synthetic glycopeptide that bears repeated ligands for the mannose 6 phosphate receptor. In some embodiments of the first and the other aspects of this disclosure, the one or more of the cell surface remodeling polymer(s) is a surfactant.

In some embodiments of the first and the other aspects of the disclosure, the cell surface remodeling polymer is a poloxamer. In some embodiments, the cell surface remodeling polymer is poloxamer p188. In some embodiments, the cell surface remodeling polymer is poloxamer p407. In some embodiments, the cell surface remodeling polymer is a combination of poloxamer p188 and poloxamer p407.

In some embodiments of the first and the other aspects of the disclosure, the one or more cell surface remodeling polymer(s) is present in the compositions of the disclosure in an amount from about 10% to about 40% by weight of the composition. In some embodiments, where the cell surface remodeling polymer is a poloxamer, the poloxamer is present in an amount from about 10% to about 40%, about 10% to about 35%, about 10% to about 25%, about 10% to 20%, about 15% to about 20%, about 15% or about 30% by weight of the composition. In some embodiments, where the cell surface remodeling polymer is a combination of poloxamer p188 and poloxamer p407, the combination is present in an amount from about 10% to about 40% or about 30% by weight of the composition. In some embodiments, where the cell surface remodeling polymer is a combination of poloxamer p188 and poloxamer p407, each poloxamer is present in an amount of 15% by weight of the composition.

In some embodiments of the first and the other aspects of the disclosure, one or more cell surface remodeling polymer(s) is present, and no optional agent(s) is present.

In some embodiments of the first and the other aspects of the disclosure, one or more optional agent(s) is present, and one or more cell surface remodeling polymer(s) is also present.

In some embodiments of the first and the other aspects of the disclosure, the optional polypropylene glycol (PPG) is present in an amount of about 0.1 to 10% by weight of the composition. In some embodiments, the optional PPG is present in an amount of about 5% to about 10% by weight, or from about 1% to about 5% by weight, or from about 0.1% to about 1% by weight of the composition.

In some embodiments of the first and the other aspects of the disclosure, one or more components of the preservative composition of this disclosure may serve the role or function of one or more of the other components of the preservative composition.

In a second aspect, the disclosure is directed to a biological marker and cell preservative composition comprising:
a. one or more enzyme inhibitors;
b. optionally one or more metabolic inhibitors;
c. one or more cell surface remodeling polymers; and
d. optionally polypropylene glycol (PPG)

The embodiments of paragraphs [0046]-[0051], [0055]-[0059] and [0060]-[0061] apply equally to the second aspect of the disclosure.

In some embodiments, this disclosure is directed to a biological marker and cell preservative composition comprising:
a. 1.57 wt % EDTA or salts thereof;
b. 15.00 wt % poloxamer p188; and
c. 15.00 wt % poloxamer p407.

In some embodiments, this disclosure is directed to a biological marker and cell preservative composition comprising:
a. 2.5 wt % EDTA or salts thereof;
b. 15.00 wt % poloxamer p188; and
c. 15.00 wt % poloxamer p407.

The preservative compositions according to the various aspects of the disclosure can be in the form of a lyophilized powder, granules, tablets, or as a solution (e.g., wherein the preservative composition is reconstituted in a suitable vehicle). The lyophilized powder, granules and/or tablets may be added directly to the biological sample or may be reconstituted prior to being added to a biological sample. The lyophilized powder, granules, and/or tablets may, for example, be reconstituted by dissolving the composition in a suitable vehicle. Suitable vehicles include but are not limited to water, saline, Ringer's solution, fixed oils of vegetable origin, mono and diglycerides of fatty acids, ethanol, glycerin, and propylene glycol. Alternatively, the biological sample may be added to the lyophilized powder, granules, tablets or the reconstituted composition (i.e., solution) directly. In some embodiments, when the biological sample is derived from a bodily fluid, the bodily fluid can serve as an acceptable vehicle for solubilizing the preservative composition. For example, the lyophilized powder, granule, and/or tablet form of the preservative composition can be combined with the bodily fluid, thereby being solubilized by the bodily fluid. In some embodiments, the collection tube or container contains the preservative composition as a lyophilized powder, granule, tablet or solution before the biological sample is collected in the tube or container.

In some embodiments, the preservative composition of the disclosure is in the form of an aqueous solution. The aqueous solution may be combined with a biological sample, or the biological sample combined with the aqueous solution.

### Combinations of a Preservative Composition and a Biological Sample and Methods of Preserving Biological Markers and/or Cells of Biological Samples

In a third aspect, the disclosure is directed to a combination of a preservative composition of the disclosure and a biological sample.

In a fourth aspect, the disclosure is directed to a method for preserving biological markers and/or cells in a biological sample comprising the steps of combining a preservative composition of this disclosure and the biological sample.

In some embodiments of the third and fourth aspects, the biological sample is a cell or tissue sample.

In some embodiments of the third and fourth aspects, the biological sample is derived from bodily fluids. In some embodiments, the bodily fluid is blood, plasma, serum, urine, saliva, stool, breast milk, tears, sweat, cerebral spinal fluid, synovial fluid, semen, vaginal fluid, ascitic fluid, or amniotic fluid. In a preferred embodiment, the biological fluid is blood, e.g., whole blood or fractions thereof. The biological sample may include cells or may be cell-free.

In some embodiments of the third and fourth aspects, the biological sample comprises stem cells, bone cells, blood cells, muscle cells, fat cells, skin cells, nerve cells, endothelial cells, sex cells, pancreatic cells, cancer cells, tumor cells, or circulating tumor cells.

In some embodiments of the third and fourth aspects, the biological sample comprises biological markers. In some embodiments, the biological markers are nucleic acids, proteins, peptides, metabolites or a combination thereof. In some embodiments, the biological markers are cellular, cell-free, or combinations thereof.

In some embodiments of the third and fourth aspects, the biological sample comprises a nucleic acid selected from RNA, DNA, or a combination thereof. In some embodiments, the nucleic acid is cell-free RNA, cell-free DNA, or a combination thereof. In some embodiments, the nucleic acid is cellular RNA, cellular DNA, or a combination thereof.

The biological sample can be combined with the preservative composition of the disclosure in a number of ways. For example, the biological sample can be collected into a suitable container followed by the addition of the preservative composition to that container, e.g., by syringe or pipette. The preservative composition can alternatively be added to a suitable container for biological sample collection prior to the collection of the biological sample. In some embodiments, the preservative composition is added to a biological sample. In some embodiments, the biological sample is added to the preservative composition.

The disclosure in these various aspects also contemplates methods wherein the components of the preservative composition are added to the biological sample simultaneously or separately. Thus, in some embodiments, the disclosure is directed to methods of preserving biological markers and/or cells in a biological sample comprising contacting a biological sample with, in any order or simultaneously, the constituent components of the preservative compositions of the disclosure. In some embodiments, a suitable container for the collection of the biological sample already contains one or more of the components of the preservative composition, and the remaining components are added to the biological sample, either sequentially, or simultaneously, with the biological sample being collected. For example, a blood collection tube already containing a suitable enzyme inhibitor (e.g., tartaric acid, or EDTA or its salts, or glucaric acid) may be used to collect the biological sample. Subsequent to the collection of the biological sample, the remaining components may be added to the biological sample. In another embodiment, the components of the preservative composition are added to the biological sample, either sequentially, or simultaneously, after the biological sample has been collected. In some embodiments, all of the required components of the preservative composition, and optionally the optional components, are present in the container before the container is used to collect the sample.

In some embodiments, the container to be used for sample collection contains the preservative composition in a lyophilized powder form. In some embodiments, the container to be used for sample collection contains the preservative composition in a granulate form. In some embodiments, the container to be used for sample collection contains the preservative composition in tablet form. In some embodiments, the container to be used for sample collection contains the preservative composition and a suitable vehicle. In some embodiments, the container to be used for sample collection contains the preservative composition as an aqueous solution. In another embodiment, the container to be used is for blood sample collection further comprises an anticoagulant. Examples of anticoagulants include but are not limited to EDTA (which may also function as an enzyme inhibitor), sodium citrate, citrate-theophylline-adenosine-dipyridamole (CTAD), lithium heparin, sodium heparin, sodium fluoride, acid-citrate-dextrode (ACD), and sodium polyanethol sulfonate. In some embodiments, the suitable container is an evacuated blood sample collection tube.

The amount of the preservative composition that may be combined with a biological sample can be determined by those skilled in the art through routine experimentation. In some embodiments, the ratio of the preservative composition to the biological sample may be from about 1:10 to about 1:1 v/v. In some embodiments, the ratio of the preservative composition to the biological sample is from about 1:8 to about 1:2 v/v. In some embodiments, the ratio of the preservative composition to the biological sample is from about 1:6 to about 1:3 v/v. In some embodiments, the ratio of the preservative composition to the biological sample is from about 1:5 to about 1:4 v/v.

After the biological samples have been collected and contacted with the preservative compositions of this disclosure, the biological markers and/or cells may be isolated from the biological sample for analysis using methods known to those skilled in the art. Such methods may include extraction, centrifugation and chromatography methods. Those skilled in the art will recognize that there are many methods that can be used to isolate the biological markers and/or cells from a biological sample.

Biological markers and/or cells that are preserved using the preservative composition of this disclosure can be isolated from treated biological samples after extended periods of storage under a variety of temperature conditions. In some embodiments, the biological sample that has been contacted with the preservative composition of this disclosure can be stored, either under ambient conditions, or low temperature for at least 1 day, at least 1 week, at least 2 weeks, at least 3 weeks or at least 4 weeks. In some embodiments, the compositions of the disclosure allow for the preservation of a biological sample (i.e., biological markers and/or cells in the biological sample) for extended periods of time at a temperature ranging from about −20° C. to about 30° C. In some embodiments, the preservative composition is capable of preserving a biological sample (i.e., biological markers and/or cells in the biological sample) for at least 1 week, at least 2 weeks, at least 3 weeks or at least 4 weeks at ambient temperature. In some embodiments the preservative composition is capable of preserving a biological sample for at least 2 weeks at ambient temperature. In some embodiments, the preservative composition of the disclosure is capable of preserving a biological sample (i.e., biological markers and/or cells in the biological sample) for at least 1 week, at least 2 weeks, at least 3 weeks or at least 4 weeks at 4° C. In some embodiments, the preservative composition of the disclosure is capable of preserving a biological sample (i.e., biological markers and/or cells in the biological sample) for at least 1 week, at least 2 weeks, at least 3 weeks or at least 4 weeks at −20° C. Nucleic acids (RNA and DNA) that are preserved using the compositions and methods of this disclosure display good yields, purity, integrity and for the RNA amplifiability.

### Kits for Preserving Biological Markers and/or Cells in a Biological Sample

The preservative compositions according to the disclosure may be provided as part of a kit that is to be received by the user. The kit allows the preservative composition(s) of this disclosure to be readily combined with a biological sample, such that the biological markers and/or the cells present in that biological sample are preserved for an extended period of time, e.g., at least 1 week, at least 2 weeks, at least 3 weeks or at least 4 weeks. The preservative composition can be provided, such that it is combined with a biological sample after that biological sample has been collected. Alternatively, the preservative composition is provided, such that it is combined with the biological sample at the time the biological sample is collected.

In some embodiments, the preservative composition is provided as an aqueous solution in a dispensing means. In some embodiments the dispensing means is a syringe. In some embodiments, the amount of preservative in the dispensing means is a predetermined amount such that the ratio of the preservative composition that is combined with the biological sample is capable of preserving the biological markers and/or cells of that sample over an extended period of time. The kit may further comprise a needle attachable to said syringe. In some embodiments, the kit is for preserving biological markers and/or cells in a blood sample, and further comprises a blood collection tube optionally containing an anticoagulant. The amount of the optional anticoagulant may be predetermined such that the collected blood sample exhibits reduced or minimal coagulation before the cells or biological markers are isolated from it. The skilled worker can readily determine these amounts using conventional methods.

In some embodiments, the preservative composition is provided in a sealed ampule, wherein said ampule comprises a removable closure, and wherein said ampule is configured to receive a dispensing means upon removal of the closure by the user. In some embodiments, the dispensing means is a pipette or a syringe. In some embodiments, the kit is for preserving biological markers and/or cells in a blood sample and further comprises a blood collection tube containing an anticoagulant.

In additional embodiments, the kit is directed to preserving biological markers and/or cells in a blood sample and comprising a blood collection tube, optionally containing a predetermined amount of an anticoagulant, and a predetermined amount of a preservative composition of this disclosure.

In a fifth aspect, the disclosure is directed to a kit for preserving biological markers and/or cells in a biological sample comprising:
a. a preservative composition disclosed herein; and
b. optionally, instructions for use of said preservative composition.

In a sixth aspect, the disclosure is directed to a kit for preserving biological markers and/or cells in a biological sample comprising:
a. a blood or other biological sample collection tube optionally containing an anticoagulant;
b. a syringe containing a preservative composition of this disclosure; and
c. optionally, a needle attachable to said syringe.

In a seventh aspect, the disclosure is directed to a kit for preserving biological markers and/or cells in a biological sample comprising:
a. a blood or other biological sample collection tube optionally containing an anticoagulant; and
b. a sealed ampule, containing a preservative of this disclosure, wherein said ampule comprises a removable closure and wherein said ampule is configured to receive a dispensing means upon removal of the closure by a user.

In some embodiments of the fifth through seventh aspects, the biological sample is a cell or tissue sample.

In some embodiments of the fifth through seventh aspects, the biological sample is derived from a bodily fluid. In some embodiments, the bodily fluid is blood, plasma, serum, urine, saliva, stool, breast milk, tears, sweat, cerebral spinal fluid, synovial fluid, semen, vaginal fluid, ascitic fluid, or amniotic fluid. In some embodiments, the bodily fluid is whole blood or fractions thereof. The biological sample may include cells or may be cell-free.

In some embodiments of the fifth through seventh aspects, the biological sample comprises stem cells, bone cells, blood cells, muscle cells, fat cells, skin cells, nerve cells, endothelial cells, sex cells, pancreatic cells, cancer cells, tumor cells, circulating tumor cells, or a combination thereof.

In some embodiments of the fifth through seventh aspects, the biological sample comprises biological markers. In some embodiments, the biological markers are nucleic acids, proteins, peptides, metabolites or a combination thereof. In some embodiments, the biological markers are cellular, cell-free, or combinations thereof.

In some embodiments of the fifth through seventh aspects, the biological sample comprises a nucleic acid selected from RNA, DNA, or a combination thereof. In some embodiments, the nucleic acid is cell-free RNA, cell-free DNA, or a combination thereof. In some embodiments, the nucleic acid is cellular RNA, cellular DNA, or a combination thereof.

### Equivalents

The foregoing description and following examples detail certain specific embodiments of the disclosure and describe the best mode of practicing this disclosure as contemplated by the inventors. It will be appreciated, however, that no matter how detailed the foregoing may appear in text, the disclosure may be practiced in many ways and the disclosure should be construed in accordance with the appended embodiments and any equivalents thereof.

Although this disclosure has been described with reference to various applications, methods, compounds, and compositions, it will be appreciated that various changes and modifications can be made without departing from the disclosure herein. The following examples are provided to better illustrate this disclosure and are not intended to limit the scope of the teachings presented herein. While the present disclosure has been described in terms of these exemplary embodiments, the skilled artisan will readily understand that numerous variations and modifications of these exemplary embodiments are possible without undue experimentation. All such variations and modifications are within the scope of the current disclosure.

### EXAMPLES

### Example 1—Compositions

The preservative compositions of this disclosure are illustrated in Table 1:

TABLE 1
Preservation compositions 1-5, 8-9 and 11-12

| Chemical | 1 | 2 | 3 | 4 | 5 | 8 | 9 | 11 | 12 |
|---|---|---|---|---|---|---|---|---|---|
| Poloxamer | 23.030% | 30.000% | 26.960% | 23.030% | 26.960% | 15.000% | 15.000% | 15.000% | 15.000% |
| p188 | | | | | | | | | |
| Poloxamer | 9.545% | — | 9.545% | 9.545% | 9.545% | 15.000% | 15.000% | 15.000% | 15.000% |
| p407 | | | | | | | | | |
| Ficoll | 1.545% | — | 1.545% | — | — | — | — | 1.500% | 3.000% |
| 2Na- | 0.706% | 1.125% | 0.710% | 0.706% | 0.710% | — | — | — | — |
| EDTA | | | | | | | | | |
| 4Na- | 0.863% | 1.375% | 0.868% | 0.863% | 0.868% | — | — | — | — |
| EDTA | | | | | | | | | |
| EDTA | 1.570% | 2.500% | 1.579% | 1.570% | 1.579% | 1.570% | 2.500% | 1.570% | 1.570% |
| (total) | | | | | | | | | |

### Example 2—Analysis of Plasma Volume

Blood samples from various donors were collected into blood sample collection tubes to assess the plasma volume of samples treated with a preservative composition according to this disclosure. The preservative compositions were tested by adding the blood sample into a tube containing 2 mL of the preservative composition.

The combined preservative composition and blood sample was then centrifuged for ˜15 minutes at room temperature and 425 g, resulting in the formation of a pellet in the collection tube. Next, without disturbing the separated components, the upper plasma layer (supernatant) was transferred to a separate collection tube using a pipette. The transferred supernatant was then centrifuged again for ˜15 minutes at 4° C. and at 16,000 g to remove any inadvertently transferred cell debris or precipitate and the volume of residual plasma is measured. The measured volume is referred to herein as the “plasma volume”.

Without wishing to be bound by theory, the “plasma volume” is expected to be an important factor in facilitating the use of the aspects of the present disclosure in high-throughput applications. The use of automation and robotics in those applications necessitates consistent plasma volumes, ideally between 3-6 mL.

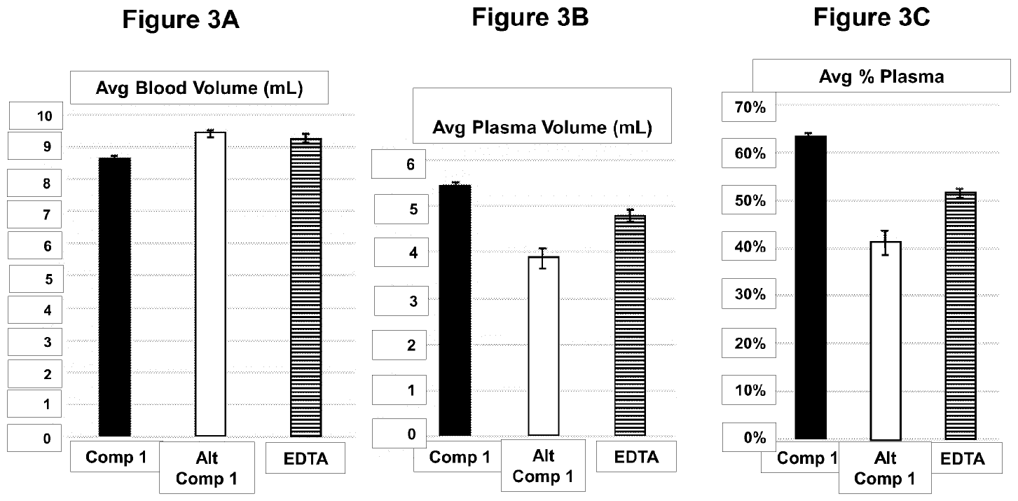

The “plasma volume” of mixtures that were processed according to the above procedure and preserved in tubes containing compositions 1-5, 8-9, and 11-12 were observed to be in the range of 4.3-4.9 mL after 2 days, 4.1-5.2 mL after 7 days, 4.2-5.0 mL after 14 days, and 4.5-5.1 mL after 21 days. All were within the desired range. See, e.g., FIGS. 3A-3C).

### Example 3—Analysis of the Integrity of the Isolated cfDNA and RNA

Blood samples from various donors were collected into the blood sample collection tubes to assess the ability of embodiments of the preservative compositions of this disclosure to preserve cfDNA and RNA. The preservative compositions were tested by adding the blood sample into a tube containing 2 mL of the preservative composition.

The cfDNA and RNA were isolated from the samples using extraction and separation techniques known in the art. One such cfDNA extraction method involves using a MagMAX™ Cell-Free DNA Isolation Kit. One such RNA extraction method involves using a procedure based on Beckman Coulter's RNAdvance Blood Kit.

The isolated cfDNA and RNA were analyzed at Day 1 and various subsequent days, the blood being drawn on Day 0. The integrity of the nucleic acids was analyzed to assess the characteristics of the preservative compositions.

### cfDNA Integrity

The integrity of the cfDNA was analyzed by qPCR of long and short DNA fragments and characterized by the ratio of long fragment over short fragment (222 bp/90 bp). The obtained ratio is referred to herein as the DNA Integrity Number (DIN). DIN is an objective metric of cfDNA quality. When the DIN is <0.5, the cfDNA is considered to be pure (i.e., the plasma is not contaminated by gDNA (cellular or genomic DNA)).

A 10-fold dilution series of the gDNA (1 ng/μL to 0.01 ng/μL) was prepared for a standard curve. A forward and reverse primer mix was prepared at 5 μM concentration by mixing 5 μL of 100 μM forward primer, 5 μL of 100 μM reverse primer with 90 μL of nuclease-free water.

Two separate mixtures were prepared with 1 μL of standard or cfDNA sample, respectively, and 8 μL of nuclease-free water for 1 reaction in mixture #1 and 1 μL of primer mixture and 10 μL of 2× PowerTrack SYBR Green Master Mix (ThermoFisher Scientific) for 1 reaction in mixture #2. 9 μL of mixture #1 and 11 μL of mixture #2 are added to the wells of a 96-well optical plate to run the real-time PCR. The thermal cycling conditions are set forth in the table below.

DNA Thermal Profile

| | | | |
|---|---|---|---|
| Cycle 1 | 95° C. | 10 min | 1X |
| Cycle 2 | 95° C. | 15 s | 40X |
| | 62° C. | 30 s | |

| | |
|---|---|
| Melting analysis | 1X |

### RNA Integrity

The integrity of RNA from the samples was analyzed by BioAnalyzer using agarose gel electrophoresis and characterized by RNA integrity number (RIN). RIN is an objective metric of total RNA quality ranging from 10 (highly intact RNA) to 1 (completely degraded RNA).

### The Properties of the cfDNA in Blood Samples Preserved Using the Compositions of Table 1

The integrity of cfDNA extracted from blood samples (i.e., the DIN) that were

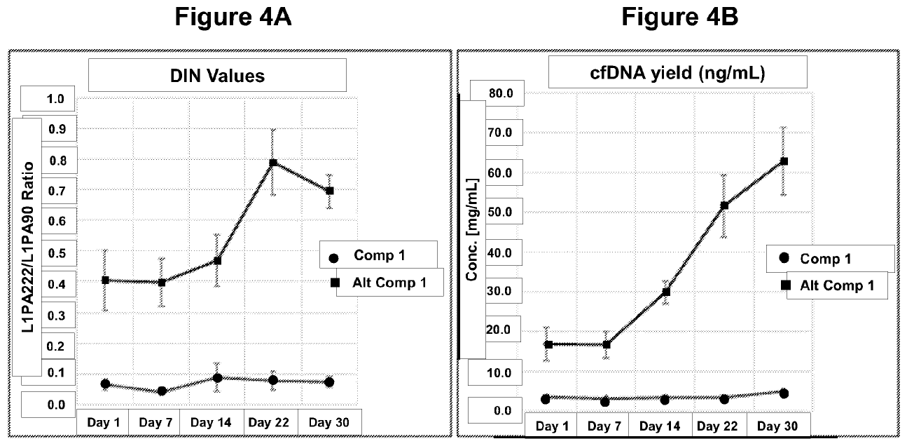

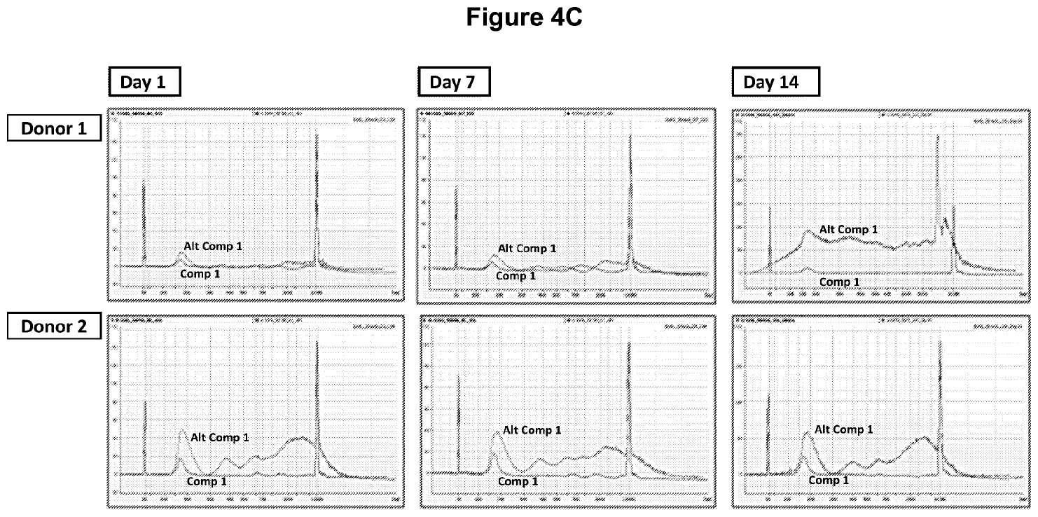

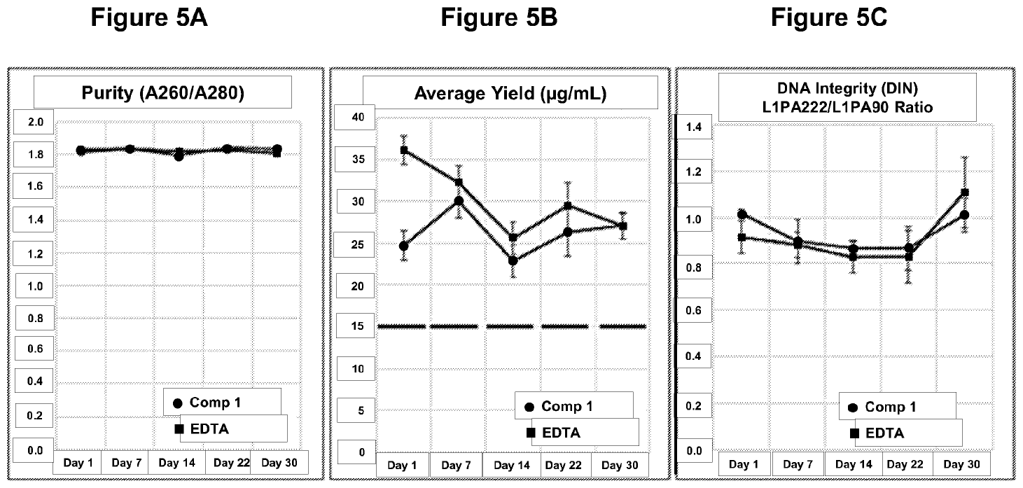

preserved in tubes containing compositions 1-5, 8-9, and 11-12 was generally high. The ratio of long fragment over short fragment (222 bp/90 bp) from the qPCR assay was observed to be in a range of <0.1-0.5 after 2 days, <0.1-0.2 after 7 days, <0.1-0.2 after 14 days and <0.1-0.21 after 21 days. See e.g., FIGS. 4A-4C and 5A-5C.

### The Properties of the RNA in Blood Samples Preserved Using the Compositions of Table 1

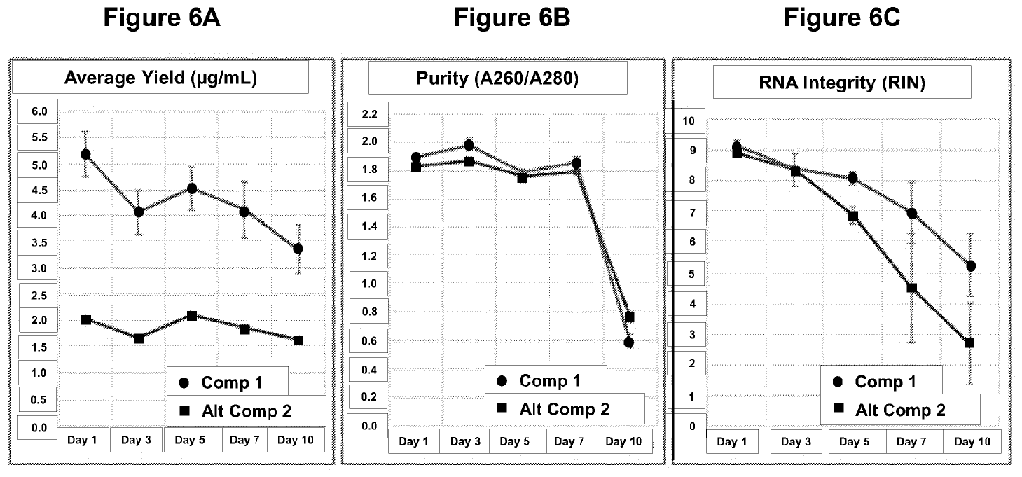

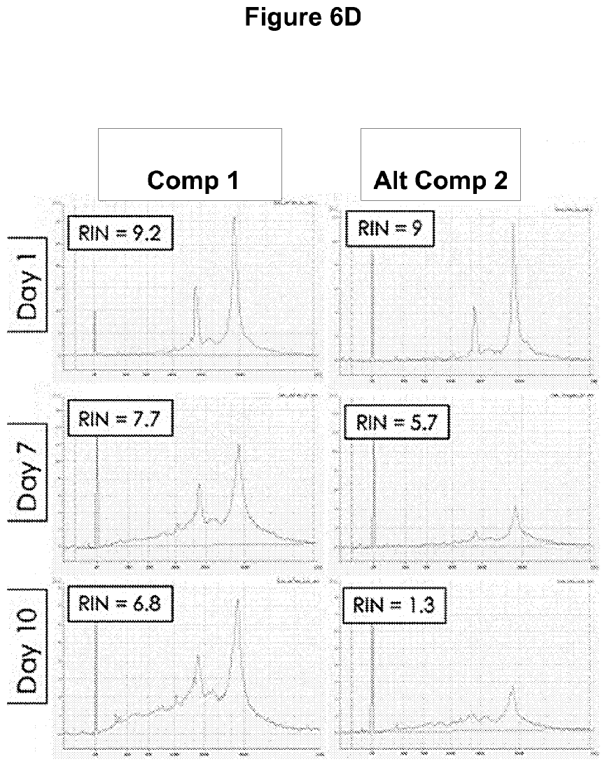

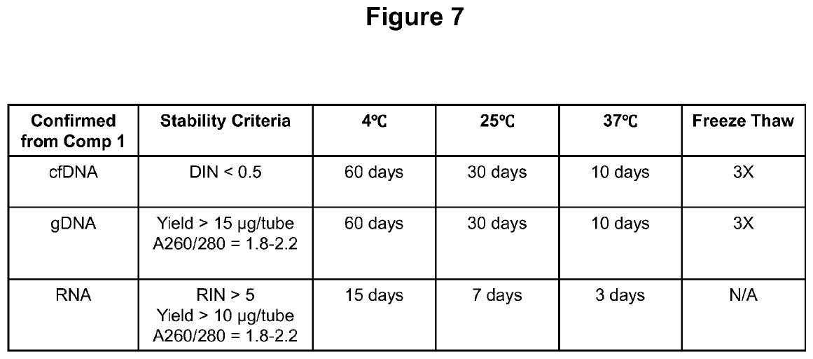

The RIN of the RNA isolated from blood samples that were preserved in tubes containing compositions 1-5, 8-9 and 11-12 was generally high, as they were observed to have a RIN in a range of 9.3-8.8 on day 2, 8.8-8.4 on day 3, 8.2-7.6 on day 5, 8.0-6.9 on day 5 7, and 7.4-5.2 on day 10. See e.g., FIGS. 6A-6D.

1. A preservative composition comprising:
a. one or more enzyme inhibitors;
b. optionally one or more metabolic inhibitors;
c. optionally one or more cell surface remodeling polymers;
d. optionally one or more agents selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them; and
e. optionally polypropylene glycol (PPG);
wherein at least one cell surface remodeling polymer (c) or agent (d) is present.

2. The preservative composition according to claim 1, wherein no osmotic agent is present.

3. The preservative composition according to any one of claim 1 or 2, wherein no plasma expander is present.

4. The preservative composition according to any one of claims 1-3, wherein the one or more optional cell surface remodeling polymer(s) is present.

5. The preservative composition according to any one of claims 1-3, wherein the one or more optional agent(s) is not present.

6. The preservative composition according to any one of claims 1-5, wherein the one or more enzyme inhibitor(s) is present in the preservative compositions in an amount of about 0.5% to about 30%, about 1% to about 10%, about 1.5% to about 5%, or about 1.5% to about 2.5% by weight of the composition.

7. The preservative composition according to claim 6, wherein the one or more enzyme inhibitor(s) is selected form the group consisting of ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, and glucaric acid, or salts of any of them.

8. The preservative composition according to claim 7, wherein the enzyme inhibitor is ethylenediaminetetraacetic acid (EDTA) or salts thereof.

9. The preservative composition according to claim 7, wherein the EDTA or salts thereof is present in an amount of about 1.5% to about 2.5% by weight of the composition.

10. The preservative composition according to any one of claims 1-9, wherein the one or more optional metabolic inhibitor(s) is sodium azide, thimerosal, proclin or chlorohexidine.

11. The preservative composition according to claims 1-4 and 6-10, wherein the one or more optional agent(s) is a Ficoll.

12. The preservative composition according to claim 11, wherein the Ficoll is Ficoll 400.

13. The preservative composition according to any one of claims 1-12, wherein the optional cell surface remodeling polymer is a surfactant.

14. The preservative composition according to claim 13, wherein the surfactant is a poloxamer.

15. The preservative composition according to claim 14, wherein the poloxamer is selected from one or both of poloxamer p188 and poloxamer p407.

16. The preservative composition according to claim 15, wherein the poloxamer is poloxamer p188.

17. The preservative composition according to claim 15, wherein the poloxamer is poloxamer p407.

18. The preservative composition according to claim 15, wherein the poloxamer is a combination of poloxamer p188 and poloxamer p407.

19. The preservative composition according to any one of claims 14-18, wherein the poloxamer(s) is present in an amount from about 10% to about 40%, about 10% to about 35%, about 10% to about 25%, about 10% to about 20%, about 15% to about 20%, or about 15% by weight or 30% by weight of the composition.

20. The preservative composition according to claim 19, wherein each of the poloxamer p188 and poloxamer p407 are present in an amount of about 15% by weight of the composition.

21. The preservative composition according to any one of claims 1-20, wherein the optional polypropylene glycol (PPG) is present in an amount of about 0.1% to about 10% by weight of the composition.

22. A preservative composition comprising:
a. one or more enzyme inhibitors;
b. optionally one or more metabolic inhibitors;
c. one or more cell surface remodeling polymers; and
d. optionally polypropylene glycol (PPG).

23. The preservative composition according to claim 22, wherein no osmotic agent is present.

24. The preservative composition according to any one of claim 22 or 23, wherein no plasma expander is present.

25. The preservative composition according to any one of claims 22-24 where no agent is selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them is present.

26. The preservative composition according to any one of claims 22-25, wherein the one or more enzyme inhibitor(s) is present in the preservative compositions in an amount of about 0.5% to about 30%, about 1% to about 10%, about 1.5% to about 5%, or about 1.5% to about 2.5% by weight of the composition.

27. The preservative composition according to claim 26, wherein the one or more enzyme inhibitor(s) is selected form the group consisting of ethylenediaminetetraacetic acid (EDTA), hydroxyethylethylenediaminetriacetic acid (HEDTA), dithiothreitol (DTT), ethylene glycol-bis(β-aminoethyl ether)-N,N,N′,N′-tetraacetic acid (EGTA), citric acid, oxalate, aurintricarboxylic acid (ATA), tartaric acid, and glucaric acid, or salts of any of them.

28. The preservative composition according to claim 27, wherein enzyme inhibitor is ethylenediaminetetraacetic acid (EDTA) or salts thereof.

29. The preservative composition according to claim 28, wherein the EDTA or salts thereof is present in an amount of about 1.5% to about 2.5% by weight of the composition.

30. The preservative composition according to claim 22-29, wherein the one or more optional metabolic inhibitor(s) is sodium azide, thimerosal, proclin or chlorohexidine.

31. The preservative composition according to claim 22-30, wherein the cell surface remodeling polymer is a surfactant.

32. The preservative composition according to claim 31, wherein the surfactant is a poloxamer.

33. The preservative composition according to claim 32, wherein the poloxamer is selected from one or both of poloxamer p188 and poloxamer p407.

34. The preservative composition according to claim 33, wherein the poloxamer is poloxamer p188.

35. The preservative composition according to claim 33, wherein the poloxamer is poloxamer p407.

36. The preservative composition according to claim 33, wherein the poloxamer is a combination of poloxamer p188 and poloxamer p407.

37. The preservative composition according to claim 33-36, wherein the poloxamer(s) is present in an amount from about 10% to about 40%, about 10% to about 35%, about 10% to about 25%, about 10% to about 20%, about 15% to about 20%, or about 15% by weight or 30% by weight of the composition.

38. The preservative composition according to claim 37, wherein each of the poloxamer p188 and poloxamer p407 are present in an amount of about 15% by weight in the composition.

39. The preservative composition according to any one of claims 22-38, wherein the optional polypropylene glycol (PPG) is present in an amount of about 0.1% to about 10% by weight of the composition.

40. A preservative composition comprising:
a. 1.57 wt % EDTA or salts thereof;
b. 15.00 wt % poloxamer p188; and
c. 15.00 wt % poloxamer p407.

41. A preservative composition comprising:
a. 2.5 wt % EDTA or salts thereof;
b. 15.00 wt % poloxamer p188; and
c. 15.00 wt % poloxamer p407.

42. The preservative composition according to any one of claim 40 or 41, wherein no osmotic agent, plasma expander or an agent selected from the group consisting of hydroxyethylstarch, a polymer of N-vinylpyrollidone (NVP), a Ficoll, a protein colloid, a non-protein synthetic colloid, ethylene diol, propylene glycol, a water-soluble polymer and carboxymethylcellulose or a salt of any of them is present.

43. The preservative composition according to any one of claims 1-42, that is in the form of a lyophilized dry powder.

44. The preservative composition according to any one of claims 1-42, that is in the form of an aqueous solution.

45. A combination of a preservative composition according to any one of claims 1-44 and a biological sample.

46. The combination according to claim 45, wherein the biological sample is a cell or tissue sample.

47. The combination according to claim 45, wherein the biological sample is derived from bodily fluids.

48. The combination according to claim 47, wherein the bodily fluid is whole blood or one of more fractions thereof.

49. The combination according to claim 45, wherein the biological sample comprises stem cells, bone cells, blood cells, muscle cells, fat cells, skin cells, nerve cells, endothelial cells, sex cells, pancreatic cells, cancer cells, tumor cells, or circulating tumor cells.

50. The combination according to claim 45, wherein the biological sample comprises a biological marker selected from a nucleic acid, a protein, a peptide, a metabolite, or a combination thereof.

51. The combination according to claim 50, wherein the biological sample comprises a nucleic acid selected from RNA, DNA, or a combination thereof.

52. The combination according to claim 51, wherein the nucleic acid is cell-free RNA, cell-free DNA, or a combination thereof.

53. The combination according to claim 51, wherein the nucleic acid is cellular RNA, cellular DNA, or a combination thereof.

54. The combination according to any one of claims 45-53, wherein the ratio of the preservative composition to the biological sample is from about 1:10 to about 1:1 v/v.

55. The combination according to claim 54, wherein the ratio of the preservative composition to the biological sample is from about 1:5 to about 1:4 v/v.

56. The combination according to any one of claims 49-55, wherein the composition is capable of preserving the nucleic acids and/or cells in a biological sample for at least 2 weeks at ambient temperature.

57. A method for preserving one or both of biological markers and cells in a biological sample comprising the steps of combining a preservative composition according to any one of claims 1-44 and the biological sample.

58. The method according to claim 57, wherein the biological sample is a cell or tissue sample.

59. The method according to claim 57, wherein the biological sample is derived from bodily fluids.

60. The method according to claim 59, wherein the bodily fluid is whole blood or one or more fractions thereof.

61. The method according to claim 57, wherein the biological sample comprises stem cells, bone cells, blood cells, muscle cells, fat cells, skin cells, nerve cells, endothelial cells, sex cells, pancreatic cells, cancer cells, tumor cells, or circulating tumor cells.

62. The method according to claim 57, wherein the biological sample comprises a biological marker selected from a nucleic acid, a protein, a peptide, a metabolite, or a combination thereof.

63. The method according to claim 62, wherein the biological sample comprises a nucleic acid selected from RNA, DNA, or a combination thereof.

64. The method according to claim 63, wherein the nucleic acid is cell-free RNA, cell-free DNA, or a combination thereof.

65. The method according to claim 63, wherein the nucleic acid is cellular RNA, cellular DNA, or a combination thereof.

66. The method according to any one of claims 57-65, wherein the ratio of the preservative composition to the biological sample is from about 1:10 to about 1:1 v/v.

67. The method according to claim 66, wherein the ratio of the preservative composition to the biological sample is from about 1:5 to about 1:4 v/v.

68. A kit for preserving one or both of biological markers and cells in a biological sample comprising:
a. a preservative composition according to any one of claims 1-44; and
b. optionally, instructions for use of said preservative composition.

69. The kit according to claim 68, wherein the biological sample is a cell or tissue sample.

70. The kit according to claim 68, wherein the biological sample is derived from a bodily fluid.

71. The kit according to claim 70, wherein the bodily fluid is whole blood or one or more fractions thereof.

72. The kit according to claim 68, wherein the biological sample comprises stem cells, bone cells, blood cells, muscle cells, fat cells, skin cells, nerve cells, endothelial cells, sex cells, pancreatic cells, cancer cells, tumor cells, circulating tumor cells, or a combination thereof.

73. The kit according to claim 68, wherein the biological sample comprises a biological marker selected from a nucleic acid, a protein, a peptide, a metabolite, or a combination thereof.

74. The kit according to claim 73, wherein the biological sample comprises a nucleic acid selected from RNA, DNA, or a combination thereof.

75. The kit according to claim 74, wherein the nucleic acid is cell-free RNA, cell-free DNA, or a combination thereof.

76. The kit according to claim 74, wherein the nucleic acid is cellular RNA, cellular DNA, or a combination thereof.

77. A kit for preserving one or both of biological markers and cells in a biological sample comprising:
a. a blood or other biological sample collection tube optionally containing a predetermined amount of an optional anticoagulant;
b. a syringe containing a predetermined amount of a preservative composition according to any one of claims 1-44; and
c. optionally, a needle attachable to said syringe.

78. The kit according to claim 77, wherein the biological sample is a cell or tissue sample.

79. The kit according to claim 77, wherein the biological sample is derived from a bodily fluid.

80. The kit according to claim 79, wherein the bodily fluid is whole blood or one or more fractions thereof.

81. The kit according to claim 77, wherein the biological sample comprises stem cells, bone cells, blood cells, muscle cells, fat cells, skin cells, nerve cells, endothelial cells, sex cells, pancreatic cells, cancer cells, tumor cells, circulating tumor cells, or a combination thereof.

82. The kit according to claim 77, wherein the biological sample comprises a biological marker selected from a nucleic acid, a protein, a peptide, a metabolite, or a combination thereof.

83. The kit according to claim 82, wherein the biological sample comprises a nucleic acid selected from RNA, DNA, or a combination thereof.

84. The kit according to claim 83, wherein the nucleic acid is cell-free RNA, cell-free DNA, or a combination thereof.

85. The kit according to claim 83, wherein the nucleic acid is cellular RNA, cellular DNA, or a combination thereof.

86. A kit for preserving one of both of biological markers and cells in a biological sample comprising:
a. a blood or other biological sample collection tube optionally containing a predetermined amount of an anticoagulant; and
b. a sealed ampule, containing a predetermined amount of a preservative composition according to any one of claims 1-44, wherein said ampule comprises a removable closure and wherein said ampule is configured to receive a dispensing means upon removal of the closure by a user.

87. The kit according to claim 86, wherein the biological sample is a cell or tissue sample.

88. The kit according to claim 86, wherein the biological sample is derived from a bodily fluid.

89. The kit according to claim 88, wherein the bodily fluid is whole blood or one or more fractions thereof.

90. The kit according to claim 86, wherein the biological sample comprises stem cells, bone cells, blood cells, muscle cells, fat cells, skin cells, nerve cells, endothelial cells, sex cells, pancreatic cells, cancer cells, tumor cells, circulating tumor cells, or a combination thereof.

91. The kit according to claim 86, wherein the biological sample comprises a biological marker selected from a nucleic acid, a protein, a peptide, a metabolite, or a combination thereof.

92. The kit according to claim 91, wherein the biological sample comprises a nucleic acid selected from RNA, DNA, or a combination thereof.

93. The kit according to claim 92, wherein the nucleic acid is cell-free RNA, cell-free DNA, or a combination thereof.

94. The kit according to claim 92, wherein the nucleic acid is cellular RNA, cellular DNA, or a combination thereof.

95. The preservative composition of any one of claims 1-44, the combination of any one of claims 45-56, the method of 57-67, or the kit of any one of claims 68-94, wherein the amount of the biological markers is maximized.

96. The preservative composition of any one of claims 1-44, the combination of any one of claims 45-56, the method of 57-67, or the kit of any one of claims 68-94, wherein the preservative composition is contained within a blood collection tube.

97. The preservative composition of any one of claims 1-44, the combination of any one of claims 45-56, the method of 57-67, or the kit of any one of claims 68-94, wherein the blood collection tube is placed into a pouch after collection and the pouch is placed into a shipping case.
